(19) United States
(12) Patent Application Publication
(10) Pub. No.: US 2026/0060603 A1
(43) Pub. Date: Mar. 5, 2026

(54) METHOD OF EARLY PREDICTION OF SEPSIS BASED ON SINGLE TIME-POINT NON-INVASIVE VITAL SIGNS

(71) Applicant: National Yang Ming Chiao Tung University, Taipei (TW)

(72) Inventor: Albert Chihchieh Yang, Brookline (MA)

(21) Appl. No.: 18/818,951

(22) Filed: Aug. 29, 2024

(51) Int. Cl. A61B 5/00; A61B 5/0205; G06N 20/00

(52) U.S. Cl. CPC A61B 5/412; A61B 5/0205; A61B 5/7267; A61B 5/7275; G06N 20/00

(57) ABSTRACT

A method of predicting onset of sepsis for a subject comprises obtaining at least one vital sign of a subject; inputting the at least one vital sign into a predetermined sepsis prediction model; and obtaining a sepsis onset prediction produced by the predetermined sepsis prediction model based on the at least one vital sign.

### FIELD OF THE INVENTION

The present disclosure relates to the technical field of biotechnology, in particular, relates to early prediction for sepsis based on single time-point non-invasive vital signs and correlation with c-reactive protein and procalcitonin.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Sepsis, a life-threatening condition triggered by the body's response to infection, ranks among the primary causes of mortality in intensive care units (ICUs) globally. The clinical standard for diagnosing sepsis primarily involves recognizing a combination of clinical signs and symptoms, supported by laboratory tests that indicate an infection and organ dysfunction. Despite advances in medical technology, early detection of sepsis remains a significant challenge in critical care. This difficulty stems from the complex and often nonspecific nature of its presentation, which can lead to delayed diagnosis and treatment, thereby increasing the risk of adverse outcomes.

The current limitations in sepsis diagnosis underline the urgent need for innovative approaches. One such promising avenue is the development of machine learning-based algorithms. A systematic review of existing literature, according to the PRISMA guidelines, suggested the potential for early and accurate prediction could revolutionize sepsis management in ICUs. Out of 974 articles identified, 22 met the criteria for inclusion in a comprehensive analysis of machine learning-driven sepsis onset prediction in ICU patients. This review revealed a diverse array of machine learning algorithms employed to refine early sepsis prediction. However, the studies' quality varied, and the massive heterogeneity in model development, sepsis definition, prediction time windows, and outcomes made a meta-analysis impractical. Most studies (86.4%) employed an offline training scenario with horizon evaluation, while a minority (9.1%) implemented an online scenario. Notably, only two studies offered publicly accessible source code and data, highlighting a significant gap in reproducibility and comparability.

A meta-analysis of various studies indicated promising results in using machine learning for sepsis prediction, suggesting that these models may surpass traditional scoring. However, insights from meta-analysis also suggest the need for improved comparability and reproducibility in these models. A systemic review of machine learning methods for sepsis prediction proposed new evaluation criteria and feature selection techniques to enhance the performance of machine learning models. The review of real-world studies on sepsis prediction algorithms revealed their effectiveness in enhancing patient care and reducing fatalities, and the ongoing challenges in applying existing guidelines for their implementation. A recent study suggested that the deployment of machine learning model (COMPOSER) for early prediction of sepsis was associated with a significant reduction in mortality and a significant increase in sepsis bundle compliance.

These studies also identified a critical gap in the current approaches to machine learning in sepsis prediction. The low comparability and reproducibility of these studies suggest that further refinement and standardization are necessary before these models can be reliably implemented in clinical settings.

Therefore, a heretofore unaddressed need exists in the art to address the aforementioned deficiencies and inadequacies, in particular, a method which can accurately predict the onset of sepsis in ICU patients using single time-point, non-invasive vital signs.

### SUMMARY OF THE INVENTION

These and other aspects of the present invention will become apparent from the following description of the preferred embodiment taken in conjunction with the following drawings, although variations and modifications therein may be effected without departing from the spirit and scope of the novel concepts of the disclosure.

In one aspect of the invention, a method of predicting onset of sepsis for a subject comprises obtaining at least one vital sign of a subject; inputting the at least one vital sign into a predetermined sepsis prediction model; and obtaining a sepsis onset prediction produced by the predetermined sepsis prediction model based on the at least one vital sign.

In one embodiment, the least one vital sign is obtained through a non-invasive method.

In one embodiment, the least one vital sign comprises at least one of heart rate, temperature, oxygen saturation, systolic blood pressure, mean arterial pressure, diastolic blood pressure, and respiration rate.

In one embodiment, the predetermined sepsis prediction model is established by a sepsis prediction modeling procedure.

In one embodiment, the sepsis prediction modeling procedure comprises training the predetermined sepsis prediction model with a training dataset comprising non-invasive vital signs of multiple training subjects at one time point; and validating the predetermined sepsis prediction model with a validating dataset comprising non-invasive vital signs of multiple validating subjects at one time point.

In one embodiment, the step of training comprises labeling each of the non-invasive vital signs of the multiple training subjects at one time point as either 0 for no-sepsis or 1 for sepsis.

In one embodiment, the step of training further comprises applying the labeled training dataset to a machine learning algorithm to obtain the predetermined sepsis prediction model.

In one embodiment, the step of training comprises further merging at least one of electronic health records and laboratory tests of each of the multiple training subjects into the predetermined sepsis prediction model; and wherein the electronic health records comprise vasopressor administration records.

In one embodiment, the machine learning algorithm is Extreme Gradient Boosting.

In one embodiment, the step of validating comprises calculating at least one key metric for the predetermined sepsis prediction model; and refining the predetermined sepsis prediction model when the at least one key metric is not within a predetermined range.

In one embodiment, the at least one key metric comprises one or more of a precision value, a recall value, and a F1 score.

In another aspect of the invention, a non-transitory computer readable medium stores a program causing a computer to execute a process for predicting onset of sepsis for a subject. The process comprises obtaining at least one vital sign of a subject; inputting the at least one vital sign into a predetermined sepsis prediction model; and obtaining a sepsis onset prediction produced by the predetermined sepsis prediction model based on the at least one vital sign.

In one embodiment, the least one vital sign is obtained through a non-invasive method.

In one embodiment, the least one vital sign comprises at least one of heart rate, temperature, oxygen saturation, systolic blood pressure, mean arterial pressure, diastolic blood pressure, and respiration rate.

In one embodiment, the predetermined sepsis prediction model is established by a sepsis prediction modeling procedure.

In one embodiment, the sepsis prediction modeling procedure comprises training the predetermined sepsis prediction model with a training dataset comprising non-invasive vital signs of multiple training subjects at one time point; and validating the predetermined sepsis prediction model with a validating dataset comprising non-invasive vital signs of multiple validating subjects at one time point.

In one embodiment, the step of training comprises labeling each of the non-invasive vital signs of the multiple training subjects at one time point as either 0 for no-sepsis or 1 for sepsis.

In one embodiment, the step of training further comprises applying the labeled training dataset to a machine learning algorithm to obtain the predetermined sepsis prediction model.

In one embodiment, the step of training comprises further merging at least one of electronic health records and laboratory tests of each of the multiple training subjects into the predetermined sepsis prediction model; and wherein the electronic health records comprise vasopressor administration records.

In one embodiment, the machine learning algorithm is Extreme Gradient Boosting.

In one embodiment, the step of validating comprises calculating at least one key metric for the predetermined sepsis prediction model; and refining the predetermined sepsis prediction model when the at least one key metric is not within a predetermined range.

In one embodiment, the at least one key metric comprises one or more of a precision value, a recall value, and a F1 score.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows a chart of Chi-squared correlation analysis of AI-predicted sepsis with biomarkers CRP and PCT.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the FIGURE. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

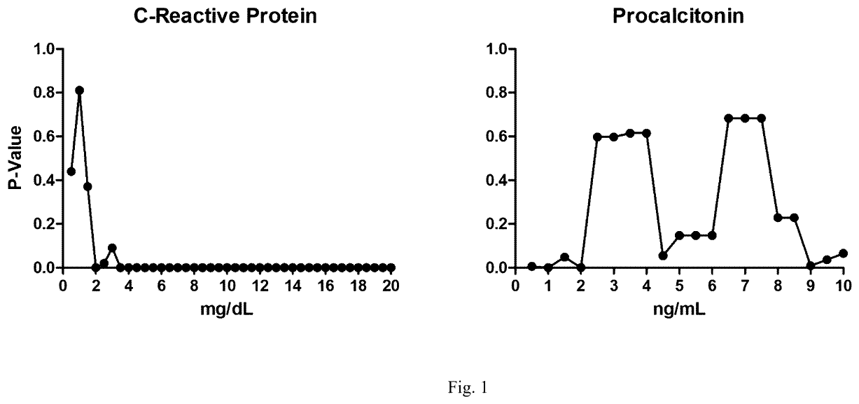

The description will be made as to the embodiments of the present invention in conjunction with the accompanying drawings in FIG. 1.

The present invention is directed to a machine learning algorithm that can accurately predict the onset of sepsis in ICU patients using single time-point, non-invasive vital signs. Machine learning algorithms for sepsis detection uses of non-invasive vital signs to predict sepsis. Vital signs, such as heart rate, blood pressure, oxygen saturation, respiration rate, and temperature, are routinely monitored in ICUs and can provide early indicators of physiological changes associated with sepsis. Machine learning algorithms of the present invention analyze these vital signs in real-time, identifying subtle changes that precede clinical recognition of sepsis. Thus, the present invention aligns with the goal of non-invasive, point-of-care monitoring, reducing the need for invasive procedures while enhancing patient care.

In one embodiment, the present invention includes a comprehensive dataset of vital signs and other relevant clinical data from Physionet and four medical centers in Taiwan, including Taipei Veterans General Hospital, Cheng-Hsin General Hospital, National Yang Ming Chiao Tung University Hospital, and Mennonite Christian Hospital. These datasets were used to train and test various machine learning models, with a focus on developing a general model for prediction of sepsis across different sites. Additionally, in one embodiment, the present invention incorporated the analysis of procalcitonin (PCT) and C-reactive protein (CRP) to evaluate the correlation between the AI predictions for sepsis and related biomarkers.

The present invention can be integrated into clinical practice, providing a tool for early sepsis detection that can inform and enhance patient management, potentially saving lives by enabling timely and targeted interventions.

### EXAMPLE

### Materials and Methods

### ICU Data

The data utilized in the present invention comprises ICU records from a large public database, Physionet, and four medical centers in Taiwan, including Taipei Veterans General Hospital (inclusion period: January 2016 to December 2021), Cheng-Hsin General Hospital (May 2017 to September 2022), National Yang Ming Chiao Tung University Hospital (January 2016 to November 2022), and Mennonite Christian Hospital (October 2019 to January 2023). These datasets provide comprehensive electronic health record information. Patient confidentiality and privacy are maintained throughout the study, with all patient identifiers removed or anonymized. This research has been conducted in accordance with ethical standards and was approved by the institutional review board of each hospital.

The initial step in data processing involves identifying all patient IDs with a sepsis label (y=1) at the base time (t=0) according to Sepsis-3. Following this identification, in one embodiment, the present invention divided the patient ID list into training, validation, and test datasets. This split was executed ensuring no duplication of patients across the datasets, thereby maintaining the integrity of the model's evaluation.

In one embodiment, the training dataset was designed to help the model learn the distinct patterns associated with sepsis. The numbers of patients in the training dataset were as follows: PhysioNet (N=1,653), Taipei Veterans General Hospital (N=437), National Yang Ming Chiao Tung University Hospital (N=87), Mennonite Christian Hospital (N=174).

In one embodiment, the validation dataset, used to fine-tune the model parameters, follows a ratio of approximately 3:1 in comparison to the training data. This dataset allows for the assessment and adjustment of the model before final evaluation. The patient numbers for validation were: PhysioNet (N=550), Taipei Veterans General Hospital (N=217), National Yang Ming Chiao Tung University Hospital (N=28), Mennonite Christian Hospital (N=57).

In one embodiment, the test dataset was for evaluating the model's final performance. In one embodiment, this dataset includes data from Cheng-Hsin General Hospital as independent cohort, which was not used in training and validation process. The patient counts for the test dataset were: PhysioNet (N=2,351), Taipei Veterans General Hospital (N=1,190), National Yang Ming Chiao Tung University Hospital (N=59), Mennonite Christian Hospital (N=352), and Cheng-Hsin General Hospital (N=80).

### Sepsis Labelling

The sepsis was defined according to the Sepsis-3 guidelines, i.e., a two-point change in the patient's Sequential Organ Failure Assessment (SOFA) score and clinical suspicion of infection (as defined by the ordering of blood cultures or IV antibiotics). Following the guideline, the present invention, in one embodiment, labelled each data row as either ‘0’ (no sepsis) or ‘1’ (sepsis) using the following procedure.

In one embodiment, the first step involved merging vital signs data with electronic health records and laboratory tests, which were routinely monitored in ICU patients. These tests were merged with the physiological data, ensuring that each data point reflects the patient's status within a 3-day window. In one embodiment, drug data, particularly the administration of vasopressors such as Dopamine, Dobutamine, Epinephrine, and Norepinephrine, indicated significant cardiovascular support, which could be associated with sepsis. In the dataset, the present invention determined if any of these drugs were administered in each row of historical data. This information provided an additional layer of context when assessing the likelihood of sepsis, alone with the blood culture or IV antibiotics.

For each data point labeled as ‘1’ (indicating sepsis), the present invention identified and labelled the data from the preceding 1 to 6 hours (t-1 to t-6) as ‘1’ as well. This approach was based on the understanding that physiological changes indicative of sepsis occur before its clinical recognition. Labeling these preceding hours as positive for sepsis (y=1) could enable the model to learn patterns that occur in the lead-up to a sepsis diagnosis. The remaining data points were kept as ‘0’ (no sepsis), as they do not fall within the critical window preceding a sepsis event. This study also sampled data points where y=0 within t-7 to t-16 hours during the training. This specific sampling was intended to help the model identify the optimal boundary between sepsis and non-sepsis states.

### Data Preprocessing

The data preprocessing involved the missing data. Specifically, any row in the raw dataset that contains missing data in any of the aforementioned features were removed. This approach was chosen to avoid introducing bias or inaccuracies that can arise from imputing missing values, especially given the critical nature of the data and the need for high precision in clinical applications.

### Machine Learning

XGBoost, short for Extreme Gradient Boosting, is an ensemble machine learning algorithm known for its efficiency and effectiveness, particularly in classification tasks. It operates as an ensemble of decision trees, applying the principle of boosting, where each new tree is built to correct the errors made by the previous ones. In one embodiment of the present invention, the demographic and non-invasive vital signs features chosen for the XGBoost model included age, sex, heart rate, temperature, oxygen saturation, systolic blood pressure, mean arterial pressure, diastolic blood pressure, and respiratory rate. These features are routine in assessing a patient's physiological state and have been identified as significant indicators for sepsis. In another embodiment, other non-invasive vital signs may be selected, either in combination with the aforementioned features, or independent of the aforementioned features, for prediction of sepsis.

The labels for model of the present invention were binary: ‘0’ (no sepsis) and ‘1’ (sepsis). To optimize the XGBoost model for sepsis prediction, the present invention implemented a grid search approach to find the optimal parameter values. This process involved systematically working through multiple combinations of parameter values and determining which combination yielded the best performance based on precision, recall, and the F1 score.

In one embodiment, the key parameters in the grid search included: (1) scale_pos_weight: Set to 1/1.4, it helped in balancing the dataset, especially when dealing with imbalanced classes, which is often the case in medical datasets; (2) max_depth: Set to 2, controlled the maximum depth of a tree and prevents overfitting; (3) reg_alpha (L1 regularization term): Set to 0.7, it added regularization to the model and reduces overfitting; (4) reg_lambda (L2 regularization term): Also set to 0.7, further contributing to the model's generalization; (5) learning_rate: Set to 0.01, this parameter determined the step size at each iteration while moving towards a minimum of a loss function; (6) min_child_weight: Set to 2, it determined the minimum sum of weights of all observations required in a child and is used to control overfitting; (7) subsample: Set at 0.8, it denoted the fraction of observations to be randomly sampled for each tree, which prevents overfitting.

### Statistical Analysis

Continuous variables were presented as mean and standard deviation, while categorical variables were expressed as numbers and percentages. In the statistical analysis of the XGBoost machine learning model for sepsis prediction, the present invention focused on three key metrics: precision, recall, and the F1 score. Precision measures the proportion of true positive predictions among all positive predictions made by the model. For this study, the aim was to achieve a precision greater than 0.5, ensuring that a majority of the sepsis cases identified by the model were indeed accurate. Recall, or sensitivity, is equally crucial as it quantifies the proportion of actual positive cases of sepsis correctly identified. A recall higher than 0.7 was targeted, underlining the model's ability to capture most true sepsis cases, which is vital in a clinical setting. The F1 score, a harmonic mean of precision and recall, balances both metrics and is especially important given the imbalanced nature of medical datasets. An F1 score above 0.6 was targeted in this study.

During the validation phase, the model's effectiveness was gauged against these metrics on the validation dataset. Failing to meet these stringent thresholds—precision over 0.5, recall above 0.7, and an F1 score exceeding 0.6—would necessitate a reevaluation and refinement of the model. Such analysis ensured that the model aligned with the critical requirements of medical applications, particularly in the accurate and reliable prediction of sepsis in ICU settings.

Finally, in one embodiment, the present invention evaluated the correlation between sepsis predictions made by the AI model and sepsis-related biomarkers, specifically PCT and CRP. PCT is a specific biomarker for the detection of sepsis, known to increase significantly in bacterial infections and sepsis, making it a valuable tool for diagnosis. On the other hand, CRP is a sensitive biomarker for inflammation, widely used in clinical settings to detect inflammatory states, including those caused by infection and tissue injury. The rapid elevation of CRP levels in response to inflammation makes it a useful marker for assessing the severity and progression of septic conditions.

In one embodiment, the present invention employed the chi-square test to evaluate the association between sepsis predictions and PCT or CRP levels above a given threshold. A p-value less than 0.05 was considered indicative of a significant association.

### Results

### Demographic Data

ICU patient data was collected from Physionet and four medical institutions in Taiwan, encompassing a total of 46,184 patients. The distribution of patients across these datasets was as follows: Physionet had the largest cohort with 39,286 patients, followed by Taipei Veterans General Hospital with 4,453 patients. Mennonite Christian Hospital accounted for 1,765 patients, Cheng-Hsin General Hospital for 1,378 patients, and National Yang Ming Chiao Tung University Hospital for 680 patients. The percentage of patients with sepsis was as follows: Physionet (N=2,746, 7.0%), Taipei Veterans General Hospital (N=726, 16.3%), Mennonite Christian Hospital (N=288, 16.3%), Cheng-Hsin General Hospital (N=12, 0.9%), and National Yang Ming Chiao Tung University Hospital (N=143, 21.0%).

To address the issue of data imbalance between sepsis and non-sepsis records, as well as the varying characteristics of the hospitals and number of patients across different sites, the present invention, in one embodiment, adopted approaches that involve randomly resampling the dataset. Table 1 presents the refined dataset with patient demographics for sepsis prediction in ICUs across these five datasets, totaling 7,235 patients. As a result, a relatively balanced dataset was achieved for both sepsis and non-sepsis single time-point records for training, validation, and testing purposes (refer to Table 2). The average age of patients varied notably across these institutions, ranging from 61.7 years at Physionet to 76.9 years at Cheng-Hsin General Hospital. The sex distribution across the five sites did not show significant differences, with the proportion of female patients ranging from 37.9% at National Yang Ming Chiao Tung University Hospital to 46.2% at Cheng-Hsin General Hospital. The incidence of sepsis in the refined datasets also varied, being highest at Physionet (60.5%) and lowest at Cheng-Hsin General Hospital (15.0%).

### Sepsis Prediction Model

Table 2 displays the performance of sepsis prediction models across five datasets, evaluated during the training, validation, and test phases. The model was trained and validated using a combination of data from Physionet, Taipei Veterans General Hospital, National Yang Ming Chiao Tung University Hospital, and Mennonite Christian Hospital, and was tested on hold-out data from the aforementioned four sites as well as an independent dataset from Cheng-Hsin General Hospital.

For Physionet, the model showed a balanced performance in terms of recall and precision during training and validation. However, in the test phase, there was a noticeable shift, with a decline in precision to 0.534, despite maintaining a recall of 0.768. Taipei Veterans General Hospital's model demonstrated notable strength in recall at 0.966 in the test phase, coupled with a precision of 0.569, underlining its efficacy in accurately identifying sepsis cases. The model at National Yang Ming Chiao Tung University Hospital maintained consistent performance, with a test phase recall of 0.841 and precision of 0.600. Similarly, Mennonite Christian Hospital's model exhibited a high recall of 0.979 in the test phase, balanced with a precision of 0.596. For the independent dataset from Cheng-Hsin General Hospital, the model achieved a recall of 0.986 and a precision of 0.585, demonstrating its consistency in detecting sepsis cases when trained on data from the other four datasets.

Correlation with Procalcitonin and c-Reactive Protein Marker

Finally, in one embodiment, the present invention assessed the correlation between sepsis AI prediction models and related biomarkers, specifically PCT and CRP. The correlation analysis was conducted using the Chi-squared test to examine the presence of sepsis and the subsequent blood test results for PCT or CRP that exceeded a given threshold. FIG. 1 shows Chi-squared correlation analysis of AI-predicted sepsis with biomarkers CRP and PCT. Elevated levels of CRP (>3.5 mg/dL) significantly correlated with AI sepsis predictions (p=3.11×10⁻⁶). In contrast, PCT levels showed no consistent correlation, with the highest significance at levels>2 ng/mL (p=3.05×10⁻⁸). As shown in FIG. 1 and Tables 3-4, the analysis revealed a significant correlation between the AI's predictions of sepsis and the levels of CRP. Specifically, AI predictions of sepsis were consistently associated with elevated CRP levels above 3.5 (i.e., p=3.11×10⁻⁶). On the other hand, PCT, known for its specificity to bacterial infections, did not demonstrate a consistent pattern of correlation with AI predictions of sepsis, despite the most significant threshold being identified at PCT>2 (i.e., p=3.05×10⁻⁸).

### Discussion

The present invention establishes an analysis of the sepsis early prediction model based on data from a public repository and four medical institutions in Taiwan, revealing noteworthy insights into the effectiveness of the sepsis early prediction and their correlation with related biomarkers. The key findings of the present invention included: (1) The sepsis early prediction models demonstrated consistent performance across different datasets, with an average recall of 0.908 and precision of 0.577, indicating a capability in effectively screening true sepsis cases. (2) There was a significant correlation between the AI predictions of sepsis and elevated levels of CRP, especially when CRP levels exceed 3.5. This finding suggested that CRP is a reliable biomarker that aligns well with AI predictions of sepsis. (3) While specific to bacterial infections, PCT showed a less consistent correlation with AI sepsis predictions. These findings suggested the potential of AI models in predicting sepsis and the clinical relevance of AI prediction associated with sepsis-related biomarkers.

The evaluation of test data, a crucial phase for determining the real-world efficacy of the sepsis early prediction model, offered significant insights into their performance. The test results consistently demonstrated the models' proficiency in effectively screening true sepsis cases, as indicated by the high average recall across datasets. However, the test data also demonstrated the inherent challenge in reducing false positives, as seen in the moderate precision values.

The observed moderate precision (i.e., 0.577 or 57.7%) could be largely attributed to the inherent complexities of sepsis prediction, which involved contending with various confounding factors. In contrast to previous methods that relied on high-dimensional electronic health records or time-series data, the approach of the present invention stands out for its use of non-invasive vital sign data. This distinction not only streamlines the prediction process but also enhances the potential for broader application in clinical environments.

Moreover, the model's design to work with single time-point data is particularly advantageous in environments where rapid decision-making is essential. In many clinical scenarios, especially in resource-limited settings, continuous monitoring and collection of time series data for each patient may not be feasible. The model of the present invention circumvents this limitation by effectively utilizing available data at any given time point, thus broadening its point-of-care applicability and ease of integration into existing healthcare systems.

The role of CRP and PCT in sepsis diagnosis and prediction is a topic of ongoing research. While both biomarkers have been shown to be useful in diagnosing sepsis, with PCT demonstrating higher diagnostic accuracy and better performance in predicting severity and response to treatment, the results were not consistent across all studies. Some studies have found PCT to be a more accurate diagnostic parameter for sepsis and a better predictor of mortality, while others have suggested that CRP may still have a role in monitoring inflammatory activity and patient responses to treatment.

The role of CRP and PCT in sepsis prediction using machine learning has also been investigated. Both of two prior studies found that PCT levels were predictive of sepsis survival, with one study also suggesting the role of APACHE II scores. Other studies further emphasized the prognostic value of PCT in sepsis, such as its correlation with severity scores. However, the use of CRP and PCT in diagnosing severe sepsis was still questionable. Overall, the combination of machine learning and biomarkers such as PCT shows promise in sepsis prediction.

The present invention emphasizes the significant role of CRP and PCT in sepsis diagnosis and prediction, especially when combined with non-invasive vital sign data in a machine learning-based sepsis early prediction model. Particularly, CRP, though sometimes considered less specific, has shown its utility in monitoring inflammatory activity and patient responses to treatment. Our data suggested that the integration of biomarkers with a machine learning model utilizing non-invasive vital signs may leverage the predictive power of the AI model, particularly in its correlation with CRP levels. Such a combination offers a more comprehensive and timely workflow for healthcare professionals. Further research is needed to evaluate the synergy of biomarkers and AI based on non-invasive data in precise sepsis diagnosis and prediction.

In conclusion, the present invention marks an advancement in sepsis diagnosis and management by introducing a non-invasive, single time-point AI sepsis early prediction model, thus eliminating the need for invasive procedures and continuous data monitoring, which are often challenging in clinical settings. Its non-invasive nature may facilitate quicker and more accessible sepsis screening. Moreover, the evaluation of the correlation between our AI model's predictions and key biomarkers, such as CRP and PCT, indicated the model's clinical relevance. By combining AI algorithms with non-invasive markers for early prediction of sepsis, our approach paves the way for more precise, timely, and patient-friendly sepsis detection and management strategies, potentially improving care in emergency and critical care settings.

TABLE 1
Demographic characteristics of refined datasets used for machine learning.

| | | | National | | |
|---|---|---|---|---|---|
| | | Taipei | Yang Ming | | Cheng- |
| | | Veterans | Chiao Tung | Mennonite | Hsin |
| | | General | University | Christian | General |
| | Physionet | Hospital | Hospital | Hospital | Hospital |
| | (N = 4,554) | (N = 1,844) | (N = 174) | (N = 583) | (N = 80) |
| Age (mean ± SD) | 61.7 ± 16.6 | 67.7 ± 18.8 | 73.6 ± 18.1 | 71.9 ± 14.9 | 76.9 ± 13.0 |
| Sex (N, %) | | | | | |
| Female | 1929, 42.4% | 708, 38.4% | 66, 37.9% | 238, 40.8% | 37, 46.2% |
| Male | 2625, 57.6% | 1136, 61.6% | 108, 62.1% | 345, 59.2% | 43, 53.8% |
| Diagnosis of | | | | | |
| Sepsis (N, %) | | | | | |
| Presence | 2,753, 60.5% | 726, 39.4% | 143, 82.2% | 288, 49.4% | 12, 15.0% |
| Absence | 1,801, 39.5% | 1,118, 60.6% | 31, 17.8% | 295, 50.6% | 68, 85.0% |
| Lengths of stay | 25.0 ± 14.5 | 470.1 ± 258.9 | 360.7 ± 227.7 | 232.5 ± 200.5 | 185.1 ± 201.2 |
| (hours, mean ± | | | | | |
| SD) | | | | | |

N: number of patients;
SD: standard deviation

TABLE 2
Performance of sepsis prediction in each dataset

| | | | National | | |
|---|---|---|---|---|---|
| | | Taipei | Yang Ming | | Cheng- |
| | | Veterans | Chiao Tung | Mennonite | Hsin |
| | | General | University | Christian | General |
| | Physionet | Hospital | Hospital | Hospital | Hospital |
| Training | | | | | |
| Sepsis (N, %) | | | | | |
| Absence | 3,561, 35.0% | 2,898, 42.3% | 150, 45.7% | 334, 45.8% | NA |
| Presence | 6,612, 65.0% | 3,946, 57.7% | 178, 54.3% | 395, 54.2% | NA |
| Accuracy | 0.602 | 0.591 | 0.564 | 0.575 | NA |
| Recall | 0.759 | 0.930 | 0.860 | 0.939 | NA |
| Precision | 0.672 | 0.593 | 0.565 | 0.565 | NA |
| F1-Score | 0.713 | 0.724 | 0.682 | 0.705 | NA |
| Validation | | | | | |
| Sepsis (N, %) | | | | | |
| Absence | 1,142, 34.2% | 1,630, 42.7% | 56, 52.3% | 75, 42.4% | NA |
| Presence | 2,200, 65.8% | 2,186, 57.3% | 51, 47.7% | 102, 57.6% | NA |
| Accuracy | 0.592 | 0.592 | 0.505 | 0.571 | NA |
| Recall | 0.751 | 0.940 | 0.745 | 0.902 | NA |
| Precision | 0.669 | 0.591 | 0.487 | 0.582 | NA |
| F1-Score | 0.708 | 0.725 | 0.589 | 0.708 | NA |
| Test | | | | | |
| Sepsis (N, %) | | | | | |
| Absence | 2,873, 51.1% | 1,489, 52.7% | 80, 49.4% | 424, 50.3% | 83, 53.2% |
| Presence | 2,747, 48.9% | 1,335, 47.3% | 82, 50.6% | 419, 49.7% | 73, 46.8% |
| Accuracy | 0.559 | 0.638 | 0.636 | 0.660 | 0.667 |
| Recall | 0.768 | 0.966 | 0.841 | 0.979 | 0.986 |
| Precision | 0.534 | 0.569 | 0.600 | 0.596 | 0.585 |
| F1-Score | 0.630 | 0.716 | 0.701 | 0.741 | 0.735 |

N: number of single time-point record with sepsis/non-sepsis label and vital signs including systolic blood pressure, diastolic blood pressure, temperature, heart rate, respiration rate, and saturation;
SD: standard deviation

TABLE 3
P-value for the Chi-squared test for sepsis
AI predictions and C-reactive protein (CRP).

| CRP Cutoff | |
|---|---|
| Value | P-value |
| 0.5 | 0.44 |
| 1 | 0.81 |
| 1.5 | 0.37 |
| 2 | 0.00070 |
| 2.5 | 0.02 |
| 3 | 0.09 |
| 3.5 | 3.11E−06 |
| 4 | 3.11E−18 |
| 4.5 | 4.92E−19 |
| 5 | 4.09E−19 |
| 5.5 | 5.69E−18 |
| 6 | 1.76E−34 |
| 6.5 | 4.72E−36 |
| 7 | 3.24E−42 |
| 7.5 | 4.53E−46 |
| 8 | 1.00E−39 |
| 8.5 | 9.10E−46 |
| 9 | 2.19E−45 |
| 9.5 | 2.00E−50 |
| 10 | 6.00E−57 |
| 10.5 | 1.16E−53 |
| 11 | 4.84E−55 |
| 11.5 | 3.77E−63 |
| 12 | 3.77E−63 |
| 12.5 | 2.98E−67 |
| 13 | 4.46E−71 |
| 13.5 | 5.69E−70 |
| 14 | 1.33E−80 |
| 14.5 | 3.68E−96 |
| 15 | 1.21E−105 |
| 15.5 | 5.77E−105 |
| 16 | 2.16E−94 |
| 16.5 | 1.83E−87 |
| 17 | 5.41E−81 |
| 17.5 | 6.41E−70 |
| 18 | 4.58E−51 |
| 18.5 | 1.01E−33 |
| 19 | 3.11E−34 |
| 19.5 | 3.95E−09 |
| 20 | 6.17E−10 |

TABLE 4
P-value for the Chi-squared test for sepsis
AI predictions and procalcitonin (PCT).

| PCT Cutoff | |
|---|---|
| Value | P-value |
| 0.5 | 0.00650 |
| 1 | 0.00024 |
| 1.5 | 0.04826 |
| 2 | 3.05E−08 |
| 2.5 | 0.59668 |
| 3 | 0.59668 |
| 3.5 | 0.61437 |
| 4 | 0.61437 |
| 4.5 | 0.05426 |
| 5 | 0.14707 |
| 5.5 | 0.14707 |
| 6 | 0.14707 |
| 6.5 | 0.68294 |
| 7 | 0.68294 |
| 7.5 | 0.68294 |
| 8 | 0.22868 |
| 8.5 | 0.22868 |
| 9 | 0.00901 |
| 9.5 | 0.03660 |
| 10 | 0.06501 |

The foregoing description of the exemplary embodiments of the invention has been presented only for the purposes of illustration and description and is not intended to be exhaustive or to limit the invention to the precise forms disclosed. Many modifications and variations are possible in light of the above teaching.

While there has been shown several and alternate embodiments of the present invention, it is to be understood that certain changes can be made as would be known to one skilled in the art without departing from the underlying scope of the invention as is discussed and set forth above and below including claims and drawings. Furthermore, the embodiments described above and claims set forth below are only intended to illustrate the principles of the present invention and are not intended to limit the scope of the invention to the disclosed elements.

References cited in the instant application, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in the description of this invention, are incorporated herein by reference in their entireties and to the same extent as if each reference was individually incorporated by reference.

What is claimed is:

1. A method of predicting onset of sepsis for a subject, the method comprising:
obtaining at least one vital sign of a subject;
inputting the at least one vital sign into a predetermined sepsis prediction model; and
obtaining a sepsis onset prediction produced by the predetermined sepsis prediction model based on the at least one vital sign.

2. The method according to claim 1, wherein the least one vital sign is obtained through a non-invasive method.

3. The method according to claim 1, wherein the least one vital sign comprises at least one of heart rate, temperature, oxygen saturation, systolic blood pressure, mean arterial pressure, diastolic blood pressure, and respiration rate.

4. The method according to claim 1, wherein the predetermined sepsis prediction model is established by a sepsis prediction modeling procedure.

5. The method according to claim 4, wherein the sepsis prediction modeling procedure comprises:
training the predetermined sepsis prediction model with a training dataset comprising non-invasive vital signs of multiple training subjects at one time point; and
validating the predetermined sepsis prediction model with a validating dataset comprising non-invasive vital signs of multiple validating subjects at one time point.

6. The method according to claim 5, wherein the step of training comprises:
labeling each of the non-invasive vital signs of the multiple training subjects at one time point as either 0 for no-sepsis or 1 for sepsis.

7. The method according to claim 6, wherein the step of training further comprises applying the labeled training dataset to a machine learning algorithm to obtain the predetermined sepsis prediction model.

8. The method according to claim 7, wherein the step of training comprises merging at least one of electronic health records and laboratory tests of each of the multiple training subjects into the predetermined sepsis prediction model; and
wherein the electronic health records comprise vasopressor administration records.

9. The method according to claim 8, wherein the machine learning algorithm is Extreme Gradient Boosting.

10. The method according to claim 5, wherein the step of validating comprises:
calculating at least one key metric for the predetermined sepsis prediction model; and
refining the predetermined sepsis prediction model when the at least one key metric is not within a predetermined range.

11. The method according to claim 10, wherein the at least one key metric comprises one or more of a precision value, a recall value, and a F1 score.

12. A non-transitory computer readable medium storing a program causing a computer to execute a process for predicting onset of sepsis for a subject, the process comprising:
obtaining at least one vital sign of a subject;
inputting the at least one vital sign into a predetermined sepsis prediction model; and
obtaining a sepsis onset prediction produced by the predetermined sepsis prediction model based on the at least one vital sign.

13. The non-transitory computer readable medium according to claim 12, wherein the least one vital sign is obtained through a non-invasive method.

14. The non-transitory computer readable medium according to claim 12, wherein the least one vital sign comprises at least one of heart rate, temperature, oxygen saturation, systolic blood pressure, mean arterial pressure, diastolic blood pressure, and respiration rate.

15. The non-transitory computer readable medium according to claim 12, wherein the predetermined sepsis prediction model is established by a sepsis prediction modeling procedure.

16. The non-transitory computer readable medium according to claim 15, wherein the sepsis prediction modeling procedure comprises:
training the predetermined sepsis prediction model with a training dataset comprising non-invasive vital signs of multiple training subjects at one time point; and
validating the predetermined sepsis prediction model with a validating dataset comprising non-invasive vital signs of multiple validating subjects at one time point.

17. The non-transitory computer readable medium according to claim 16, wherein the step of training comprises:
labeling each of non-invasive vital signs of the multiple training subjects at one time point as either 0 for no-sepsis or 1 for sepsis.

18. The non-transitory computer readable medium according to claim 17, wherein the step of training further comprises applying the training dataset to a machine learning algorithm to obtain the predetermined sepsis prediction model.

19. The non-transitory computer readable medium according to claim 18, wherein the step of training further comprises merging at least one of electronic health records and laboratory tests of each of the multiple training subjects to the non-invasive vital signs of the multiple validating subjects; and
wherein the electronic health records comprise vasopressor administration records.

20. The non-transitory computer readable medium according to claim 19, wherein the machine learning algorithm is Extreme Gradient Boosting.

21. The non-transitory computer readable medium according to claim 16, wherein the step of validating comprises:
calculating at least one key metric for the predetermined sepsis prediction model; and
refining the predetermined sepsis prediction model when the at least one key metric is not within a predetermined range.

22. The non-transitory computer readable medium according to claim 21, wherein the at least one key metric comprises one or more of a precision value, a recall value, and a F1 score.
